SEQUENCE LISTING (5 sequences)

SEQ ID NO: 1 - RNA, 17 nt, synthetic construct
  source 1..17: mol_type = other RNA
gtaaaacgacggccagt

SEQ ID NO: 2 - RNA, 33 nt, synthetic construct
  source 1..33: mol_type = other RNA
gagctcacctgtgtgaaattgttatccgctctt

SEQ ID NO: 3 - RNA, 50 nt, synthetic construct
  source 1..50: mol_type = other RNA
gtaaaacgacggccagtgagctcacctgtgtgaaattgttatccgctctt

SEQ ID NO: 4 - RNA, 18 nt, synthetic construct
  source 1..18: mol_type = other RNA
tctttgcacgatggaatg

SEQ ID NO: 5 - RNA, 32 nt, synthetic construct
  source 1..32: mol_type = other RNA
gagctcaagcatccaaggcaactgttttttta

(19) United States
(12) Patent Application Publication
(10) Pub. No.: US 2026/0158052 A1
(43) Pub. Date: Jun. 11, 2026

(54) THERAPEUTIC AGENT, PROGRESSION INHIBITOR, AND PREVENTIVE AGENT FOR NEURODEGENERATIVE DISORDERS CHARACTERIZED BY AGGREGATION OF TDP43, TDP35, OR TDP25

(71) Applicant: NATIONAL UNIVERSITY CORPORATION HOKKAIDO UNIVERSITY, Sapporo-shi, Hokkaido (JP)

(72) Inventors: Akira KITAMURA, Hokkaido (JP); Ai FUJIMOTO, Hokkaido (JP)

(21) Appl. No.: 18/706,442

(22) PCT Filed: Nov. 2, 2022
(86) PCT No.: PCT/JP2022/041001

§ 371 Date: Nov. 13, 2024

Related U.S. Application Data
(60) Provisional application No. 63/275,408, filed on Nov. 3, 2021.

(51) Int. Cl. A61K 31/7105; A61P 25/28; C12N 15/11

(52) U.S. Cl. CPC A61K 31/7105; A61P 25/28; C12N 15/111

(57) ABSTRACT

The present invention provides an aggregation inhibitor that inhibits aggregation of TDP-43 or a partial protein thereof, the aggregation inhibitor containing, as an active ingredient, an RNA molecule having either a G4C2 sequence, which is a base sequence represented by GGGGCC, or an A4U2 sequence, which is a base sequence represented by AAAAUU; and also provides the aggregation inhibitor wherein the RNA molecule includes RNA in which from one to eight of the G4C2 sequences are linked; and a pharmaceutical composition containing one of the aggregation inhibitors as an active ingredient, wherein the pharmaceutical composition is administered to inhibit the aggregation of TDP-43 or a partial protein thereof.

Priority is claimed on U.S. Patent Application No. 63/275,408, filed in the U.S.A on Nov. 3, 2021, the content of which is incorporated herein by reference.

### TECHNICAL FIELD

The present invention relates to a compound that is useful as a therapeutic agent, a progression inhibitor and a preventive agent for neurodegenerative diseases characterized by aggregation of TDP-43 and partial proteins thereof, and also relates to a pharmaceutical composition that contains the compound as an active ingredient.

### BACKGROUND ART

One theory that has been proposed for the main cause of neuronal cell death in neurodegenerative diseases is the disruption of homeostasis by aggregates of proteins that have failed to fold. Actual pathological findings in various neurodegenerative diseases have confirmed high frequencies of protein aggregates accumulated inside neuronal cells. Molecular chaperones are already known as a protein group that inhibit this type of protein aggregate (Non-Patent Document 1). Until recently, molecular groups capable of preventing protein aggregate formation were thought to be limited to these proteins known as molecular chaperones, but in recent years, the concept that RNA may also have this type of function as a chaperone that can inhibit protein aggregate formation has been proposed.

On the other hand, the inventors of the present invention have reported that in TDP-43 (a TAR DNA/RNA binding protein of 43 kDa), which is one of the proteins that cause amyotrophic lateral sclerosis (ALS), a neurodegenerative disease that results in a loss of motor neurons, aggregate formation of a carboxyl (C) terminal fragment TDP-25 of the TDP-43 starts as a result of RNA depletion (Non-Patent Document 2). There is a possibility that the RNA is functioning as a molecular chaperone for inhibiting the aggregation of TDP-43, but it is not known specifically what type of RNA functions as this type of molecular chaperone. TDP-25 is a 25 kDa fragment that exhibits high levels of aggregation and cytotoxicity among the C-terminal fragments of TDP-43.

TDP-43 is a protein that binds to RNA/DNA, and typically known recognition sequences for TDP-43 include UG or TG repeats. However, no interaction was detected between TDP-25 and the UG/TG repeats (Non-Patent Document 2). On the other hand, TDP-43 has been reported as binding to RNA that forms a three-dimensional structure known as a guanine quadruplex (Non-Patent Documents 3 and 4). Further, the GGGGCC repeat RNA that forms this guanine quadruplex exists in the untranslated region of transcripts of the C90RF72 gene in which mutations are most frequently observed in familial ALS. In ALS patients, that repeat undergoes abnormal expansion, and it has been suggested that this makes formation of a normal guanine quadruplex impossible (Non-Patent Document 5). However, the actions of this GGGGCC repeat that exists in the untranslated region of the C90RF72 gene and the guanine quadruplex structure thereof remain completely unknown.

### CITATION LIST

### Non Patent Documents

### [Non Patent Document 1]

Shinnige et al., Advances in Experimental Medicine and Biology, 2020, vol. 1243, pp. 53 to 68.

### [Non Patent Document 2]

Kitamura et al., Scientific Reports, 2016, 6:19230

### [Non Patent Document 3]

Ishiguro et al., FEBS Letters. 2020, vol. 594, pp. 2254 to 2265.

### [Non Patent Document 4]

Ishiguro et al., Genes to Cells, 2016, vol. 21, pp. 466 to 481.

### [Non Patent Document 5]

Marogianni et al., Neurobiology of Aging 2019, vol. 84, pp. 238.e25 to 238.e34.

### [Non Patent Document 6]

Kitamura et al., International Journal of Molecular Sciences, 2015, vol. 16, pp. 6076 to 6092.

### [Non Patent Document 7]

Shodai et al., Journal of Biological Chemistry, 2013, vol. 288, pp. 14886 to 14905.

### SUMMARY OF INVENTION

### Technical Problem

The present invention has an object of providing a novel aggregation inhibitor for inhibiting TDP-43 aggregate formation, and a pharmaceutical composition that contains the aggregation inhibitor as an active ingredient.

### Solution to Problem

The inventors of the present invention discovered that a GGGGCC repeat RNA binds to TDP-43 and the C-terminal fragment TDP-25 thereof, and functions as a chaperone RNA that reduces aggregate formation in the cytoplasm, thus enabling them to complete the present invention.

In other words, the present invention includes the following aspects.
[1] An aggregation inhibitor that inhibits aggregation of TDP-43 or a partial protein thereof, the aggregation inhibitor containing, as an active ingredient, an RNA molecule having either a G4C2 sequence, which is a base sequence represented by GGGGCC, or an A4U2 sequence, which is a base sequence represented by AAAAUU.
[2] The aggregation inhibitor according to [1], wherein the RNA molecule includes RNA in which from one to eight of the G4C2 sequences are linked.
[3] The aggregation inhibitor according to [1], wherein the RNA molecule includes RNA in which from one to eight of the A4U2 sequences are linked.
[4] The aggregation inhibitor according to [1], wherein the RNA molecule contains both the G4C2 sequence and the A4U2 sequence.
[5] A pharmaceutical composition containing the aggregation inhibitor according to any one of [1] to [4] as an active ingredient, wherein the pharmaceutical composition is administered to inhibit the aggregation of TDP-43 or a partial protein thereof.
[6] The pharmaceutical composition according to [5], used in the prevention or treatment of a neurodegenerative disease.
[7] The pharmaceutical composition according to [6], wherein the neurodegenerative disease is amyotrophic lateral sclerosis or frontotemporal lobar degeneration.

### Advantageous Effects of Invention

The aggregation inhibitor according to the present invention contains, as an active ingredient, an RNA that functions as a molecular chaperone that contributes to inhibition of TDP-43 aggregation. As a result, a pharmaceutical composition containing the aggregation inhibitor according to the present invention as an active ingredient is useful in the treatment and prevention of various diseases such as ALS in which TDP-43 aggregate formation is one of the causes of the disease.

### BRIEF DESCRIPTION OF DRAWINGS

FIG. 1A is a diagram illustrating the results for RCA measured in Example 1 using a disrupted cell suspension of cells expressing GFP, GFP-TDP43 or GFP-TDP25, and AF-labeled r(G4C2)₄ that formed a Gq structure in the presence of KCl (G4K+K), AF-labeled r(G4C2)₄ prepared in the presence of LiCl (G4Li+K), AF-labeled r(A4U2)₄ prepared in the presence of KCl (A4K+K), AF-labeled r(A4U2)₄ prepared in the presence of LiCl (A4Li+K), or AF-labeled r(UG)₁₂ prepared in the presence of KCl (UG).

FIG. 1B is a diagram illustrating the results for RCA measured in Example 1 using a disrupted cell suspension of cells expressing GFP, GFP-TDP43 or GFP-TDP25, and AF-labeled r(G4C2)₄ that formed a Gq structure in the presence of KCl (Gq+HP), AF-labeled r(G4C2)₄ prepared in the presence of LiCl (G4(HP)), AF-labeled r(A4U2)₄ prepared in the presence of KCl (A4), or AF-labeled r(UG)₂ prepared in the presence of KCl (UG).

FIG. 2 is a diagram illustrating the results for RCA measured in Example 1 using a disrupted cell suspension of cells expressing GFP, GFP-TDP43 or GFP-TDP25, and AF-labeled r(G4C2)₄ that formed a Gq structure in the presence of KCl (G4K+K), AF-labeled r(G4C2)₂ that formed a Gq structure in the presence of KCl (G2K+K), AF-labeled r(G4C2)₂ prepared in the presence of LiCl (G2Li+K), AF-labeled r(A4U2)₂ prepared in the presence of KCl (A2K+K), or AF-labeled r(A4U2)₂ prepared in the presence of LiCl (A2Li+K).

FIG. 3A is a diagram illustrating the results for RCA measured in Example 1 using purified GFP, GFP-TDP43 or GFP-TDP25, and AF-labeled r(G4C2)₄, AF-labeled r(A4U2)₄ or AF-labeled r(U)₂₀.

FIG. 3B is a diagram illustrating the results for RCA measured in Example 1 using purified GFP, GFP-TDP43 or GFP-TDP25, and AF-labeled r(G4C2)₄ that formed a Gq structure in the presence of KCl, AF-labeled r(A4U2)₄ prepared in the presence of KCl, or the AF dye alone.

FIG. 4A is a diagram illustrating the results for RCA measured in Example 1 using a disrupted cell suspension of cells expressing GFP, GFP-TDP43, GFP-TDP25, GFP-CTF233, GFP-CTF263 or GFP-CTF274, and AF-labeled r(G4C2)₄ or AF-labeled r(A4U2)₄.

FIG. 4B is a diagram illustrating the results for RCA measured in Example 1 using a disrupted cell suspension of cells expressing GFP, GFP-TDP43, GFP-TDP25, GFP-CTF233 or GFP-CTF274, and AF-labeled r(G4C2)₄ that formed a Gq structure in the presence of KCl (Gq+HP), AF-labeled r(G4C2)₄ prepared in the presence of LiCl (G4(HP)), or AF-labeled r(A4U2)₄ prepared in the presence of KCl (A4).

FIG. 5 is a diagram illustrating the results, in Example 2, of measuring the intracellular aggregate positivity rate (%) in Neuro2a cells expressing GFP-TDP25 and r(M13-G4), r(M13-G2), r(M13-A4), r(M13-A2) or r(M13N).

FIG. 6 is a diagram illustrating the results, in Example 2, of measuring the intracellular aggregate positivity rate (%) in Neuro2a cells expressing GFP-TDP25 and an empty vector, r(mKeima), r(mKeima-G4) or r(mKeima-A4).

FIG. 7 is a diagram illustrating the results, in Example 2, of measuring the intracellular aggregate positivity rate (%) in Neuro2a cells transfected with pHS-GFP-TDP25 and r(G4C2)₄ or r(A4U2)₄, and in Neuro2a cells transfected only with pHS-GFP-TDP25.

FIG. 8 is a diagram illustrating the results, in Example 2, of measuring the intracellular aggregate positivity rate (%) in Neuro2a cells expressing GFP-TDP43CS and an empty vector, r(M13N), r(m13-G4) or r(M13-A4).

FIG. 9 is a diagram illustrating the results, in Example 2, of measuring the proportion (%) of round and shrunk cells among the total cell count for 293 cells expressing GFP-TDP43CS and r(m13-G4), r(M13-A4) or r(M13N), and for 293 cells expressing only GFP-TDP43CS.

### DESCRIPTION OF EMBODIMENTS

The aggregation inhibitor according to the present invention has, as an active ingredient, an RNA molecule having a G4C2 sequence, which is a base sequence represented by GGGGCC, or an A4U2 sequence, which is a base sequence represented by AAAAUU, and inhibits the aggregation of TDP-43 or partial proteins thereof. Here, “G” represents a guanine base, “C” represents a cytosine base, “A” represents an adenine base, and “U” represents an uracil base.

TDP-43 starts to undergo aggregation when the denaturation of monomers leads to conformational change. First, a plurality of denatured monomers polymerize to form an oligomer. Aggregation of these oligomers then results in aggregate formation. As illustrated in the examples below, RNA comprising the G4C2 sequence binds to the RPM2 domain of TDP-43, and particularly the amino acid region 220 to 262, and functions as a molecular chaperone, thereby directly inhibiting the oligomerization and aggregation of TDP-43 and the like. RNA comprising the A4U2 sequence exhibits little binding ability to oligomers and aggregates of TDP-43 and the like, but can inhibit TDP-43 aggregate formation by inhibiting the denaturation of TDP-43 monomers and the oligomerization of denatured monomers.

TDP-43 is a protein composed of 414 amino acids that is also known as ALS10 (ALS causative protein 10), and from the N-terminal side, has an RPM1 domain (amino acid region 106 to 176), an RPM2 domain (amino acid region 191 to 262), and a glycine-rich region (amino acid region 274 to 414). The glycine-rich region of the C-terminal is a proline-like region which participates in aggregation. C-terminal-containing fragments of TDP-43 include TDP-35 (fragment of the amino acid region 90 to 414) and TDP-25 (fragment of the amino acid region 220 to 414), and both of these fragments are aggregating proteins containing a glycine-rich region. The TDP-43 aggregation inhibitory effect of RNA comprising the G4C2 sequence and RNA comprising the A4U2 sequence manifests not only for TDP-43, but also relative to TDP-35 and TDP-25.

The RNA molecule that functions as the active ingredient of the aggregation inhibitor according to the present invention need only contain one G4C2 sequence or A4U2 sequence. For example, RNA comprising a single A4U2 sequence or RNA comprising a single G4C2 sequence can be used as the active ingredient of the aggregation inhibitor according to the present invention, or RNA containing one A4U2 sequence and one G4C2 sequence linked together may also be used as the active ingredient. In terms of achieving a more satisfactory aggregation inhibitory effect, the RNA molecule of the active ingredient of the aggregation inhibitor according to the present invention preferably has a repeat region containing a base sequence that includes two or more repeating G4C2 sequences or A4U2 sequences linked together. Among the various possibilities, RNA containing two to eight repeating G4C2 sequences linked together and RNA containing two to eight repeating A4U2 sequences linked together are preferred, and RNA containing two to four repeating G4C2 sequences linked together and RNA containing two to four repeating A4U2 sequences linked together are particularly preferred.

The RNA molecule that functions as the active ingredient of the aggregation inhibitor according to the present invention may also exist in salt form. Examples of the acid or base used in forming the salt include alkali metals such as sodium and potassium; alkaline earth metals such as calcium and magnesium; mineral acids such as hydrochloric acid and sulfuric acid; and organic acids such as acetic acid, succinic acid and citric acid. Further, the RNA molecule that functions as the active ingredient of the aggregation inhibitor according to the present invention may also exist in solvate form such as a hydrate.

In those cases where the RNA molecule that functions as the active ingredient of the aggregation inhibitor according to the present invention is an RNA molecule having the G4C2 sequence, the molecule preferably forms a complex with a potassium ion. Coordination with a potassium ion enables the RNA molecule having the G4C2 sequence to stably form a guanine quadruplex (Gq) structure. By adopting a Gq structure, the RNA molecule having the G4C2 sequence is able to generate a more powerful aggregation inhibitory effect on TDP-43.

The RNA molecule that functions as the active ingredient of the aggregation inhibitor according to the present invention may also have other base sequences besides the G4C2 sequence and A4U2 sequence, provided that the aggregation inhibitory effect provided by the G4C2 sequence or A4U2 sequence is not impaired. For example, an RNA molecule having a promoter sequence upstream (on the 5′ side) of the G4C2 sequence or A4U2 sequence may be used as the active ingredient of the aggregation inhibitor according to the present invention. Examples of promote sequences that may be used include promoters selected appropriately from among conventional promoter sequences, such as an M13 promoter, T3 promoter, SP6 promoter, CMV promoter or H1 promoter. Further, the promoter sequence and the G4C2 sequence or the like may be either linked directly, or linked via a suitable RNA linker.

In those cases where the RNA molecule that functions as the active ingredient of the aggregation inhibitor according to the present invention has another base sequence besides the G4C2 sequence or A4U2 sequence, that other base sequence may be a base sequence composed only of RNA, a base sequence composed only of DNA, or a base sequence composed of DNA and RNA. Further, one or more bases within the other base sequence may be modified. Examples of this modification include methylation, amination, methoxylation, deamination, dihydrogenation, thiolation, acetylation, and carboxylation.

The RNA molecule that functions as the active ingredient of the aggregation inhibitor according to the present invention may also be linked to a non-nucleic acid substance outside the nucleic acid portion containing the RNA, provided that the aggregation inhibitory effect provided by the G4C2 sequence or A4U2 sequence is not impaired. Examples of this other substance include peptides, proteins, sugars, lipids, and low-molecular weight molecules. The linkage between these types of other substances and the RNA containing the G4C2 sequence or A4U2 sequence may be formed using any of the various linkage methods used when modifying RNA with a peptide or the like.

The aggregation inhibitor according to the present invention is an RNA molecule of comparatively low molecular weight, and therefore does not suffer from problems of immunogenicity or the like. Further, the aggregation inhibitor can be administered orally, and because there are few limitations on the administration pathway, the compound is particularly useful as the active ingredient for pharmaceutical compositions designed for mammals including humans. These pharmaceutical compositions may contain either one type, or two or more types, of the aggregation inhibitor according to the present invention.

The aggregation inhibitor according to the present invention can inhibit the aggregation of TDP-43. As a result, the aggregation inhibitor according to the present invention can be used as a reagent for inhibiting the aggregation of TDP-43 or partial proteins thereof either in vitro or ex vivo. Furthermore, the aggregation inhibitor according to the present invention is also useful as the active ingredient of a pharmaceutical composition used in the treatment or prevention of various diseases in which TDP-43 aggregate formation is one of the causes of the disease. Examples of these diseases in which TDP-43 aggregate formation is one of the causes of the disease include neurodegenerative diseases such as amyotrophic lateral sclerosis (ALS) and frontotemporal lobar degeneration (FTLD).

In those cases where either one type, or two or more types, of the aggregation inhibitor according to the present invention are included in a pharmaceutical composition, the aggregation inhibitor(s) may be blended with a pharmaceutically acceptable carrier if necessary, and any of various dosage forms may be used depending on the purpose of the prevention or treatment. Examples of those dosage forms include oral agents, injections, suppositories, ointments and patches, although oral agents are preferred. These dosage forms can be produced using production methods well-known to those in the field.

Examples of materials that may be used as pharmaceutically acceptable carriers include excipients, binders, disintegrants, lubricants and colorants used in solid formulations; and solvents, solubilizers, suspension agents, isotonicity agents, buffers, and pain relievers and the like used in liquid formulations. Further, formulation additives such as preservatives, antioxidants, colorants, sweeteners, and stabilizers may also be used if necessary.

In the case of preparation of a solid formulation for oral administration, an excipient, and if necessary a binder, disintegrant, lubricant, colorant, and/or flavoring agent or the like are added to the nucleic acid compound according to the present invention, and a typical method may then be used to produce tablets, coated tablets, granules, powders, or capsules or the like.

In the case of preparation of a liquid formulation for oral administration, a sweetener, buffer, stabilizer, and/or flavoring agent or the like are added to the nucleic acid compound according to the present invention, and a typical method may then be used to produce an internal liquid formulation, syrup, or elixir or the like.

In the case of preparation of an injection, a pH regulator, buffer, stabilizer, isotonicity agent, and/or local anesthetic or the like are added to the nucleic acid compound according to the present invention, and a typical method may then be used to produce a subcutaneous, intramuscular, or intravenous injection.

In the case of preparation of a suppository, a formulation carrier known to those in the field, such as polyethylene glycol, lanolin, cacao butter, or a fatty acid triglyceride or the like, is added to the nucleic acid compound according to the present invention, and a typical method may then be used to produce the suppository.

In the case of preparation of an ointment, a typically used base agent, stabilizer, humectant and/or preservative or the like are added as necessary to the nucleic acid compound according to the present invention, and typical methods are then used to mix the ingredients and form the ointment.

In the case of preparation of a patch, an aforementioned ointment, or a cream, gel or paste or the like may be applied to a typical support using any typical method.

The amount of the nucleic acid compound according to the present invention within each of the formulations described above varies depending on the symptoms of the patient and the dosage form and the like, but typically, is within a range from about 0.001 to 1,000 mg in the case of an oral agent, within a range from about 0.001 to 500 mg in the case of an injection, and within a range from about 0.01 to 1,000 mg in the case of a suppository.

Furthermore, although the dosage per day for these formulations cannot be generalized, and varies depending on the symptoms, weight, age, and gender and the like of the patient, the dosage per day for a typical adult (weight: 60 kg) is within a range from about 0.005 to 5,000 mg, and preferably from 0.01 to 1,000 mg, and this dosage is preferably administered either once per day, or divided into about 2 or 3 doses per day.

There are no particular limitations on the animals that may be administered with the pharmaceutical composition containing the aggregation inhibitor according to the present invention as an active ingredient, and the animal may be either a human or a non-human animal. Examples of these non-human animals include mammals such as cows, pigs, horses, sheep, goats, monkeys, dogs, cats, rabbits, mice, rats, hamsters and marmots, and birds such as chickens, quail and ducks.

### EXAMPLES

The present invention is described below in further detail using a series of examples, but the present invention is not limited to the following examples.

### Example 1

The binding properties of TDP-43, and TDP-25 which represents an aggregating carboxyl terminal fragment of TDP-43, with RNA having a G4C2 sequence and RNA having an A4U2 sequence were investigated. As a comparison, RNA having a UG sequence was also investigated in a similar manner. RNA having a UG sequence is known to bind to TDP-43.

### <Measurement of Relative Cross-Correlation Amplitude (RCA)>

The binding properties between TDP-43 and TDP-25, and RNA were evaluated based on relative cross-correlation amplitude (RCA) values measured by fluorescence cross-correlation spectroscopy (FCCS). FCCS is a method for investigating molecular interactions in solution with single molecule sensitivity (Non-Patent Document 6), and this method was used to analyze the interactions between GFP-tagged proteins and Alexa Fluor 647-labeled (hereinafter also abbreviated as “AF-labeled”) RNA. GFP-tagged TDP-43 (GFP-TDP43) and GFP-tagged TDP-25 (GFP-TDP25) were prepared in accordance with the method disclosed in Non-Patent Document 2. RNA containing four linked repeats of the G4C2 sequence (AF-labeled r(G4C2)₄), RNA containing two linked repeats of the G4C2 sequence (AF-labeled r(G4C2)₂), RNA containing four linked repeats of the A4U2 sequence (AF-labeled r(A4U2)₄), RNA containing two linked repeats of the A4U2 sequence (AF-labeled r(A4U2)₂), RNA containing 12 linked repeats of the UG sequence (AF-labeled r(UG)₁₂), and RNA containing 20 linked U repeats (AF-labeled r(U)₂₀) were synthesized by chemical synthesis (RNA oligonucleotide synthesis service, Ajinomoto Bio-Pharma Inc.), and in each case, the 5′-terminal was labeled with AF using typical methods.

The GFP-TDP43 or GFP-TDP25 was expressed with a His tag in mouse neuroblastoma Neuro2a cells (#CCL-131, acquired from ATCC) and then purified. Culturing of the Neuro2a cells was conducted using a medium prepared by adding 10% by volume FBS (manufactured by Thermo Fisher Scientific Inc.), 100 U/mL of penicillin G (manufactured by Sigma-Aldrich Corporation) and 0.1 mg/mL of streptomycin (manufactured by Sigma-Aldrich Corporation) to DMEM (manufactured by Sigma-Aldrich Corporation). A plasmid solution was prepared by dissolving a mixture of 14 μg of salmon sperm DNA (manufactured by BioDynamics Laboratory Inc.) and 16 μg of an expression plasmid DNA (pHS-GFP-TDP43, pHS-GFP-TDP25, or pHS-GFP) in a 150 mM aqueous solution of NaCl, subsequently adding a solution prepared by dissolving 77.5 pg of polyethyleneimine (PEI) in a 10 mM Tris-HCl buffer (pH 8.0), and then incubating the mixture for 15 minutes. The thus obtained plasmid solution was added to dishes containing the cultured Neuro2A cells (3.0×10⁶ cells/150 mm dish) and the dishes were incubated for 24 hours to express proteins having both an His tag and a GFP tag.

Following incubation, the cells were trypsinized and collected in a tube, and with the cells cooled over ice, a lysis buffer (a buffer containing 50 mM HEPES-KOH (pH 7.5), 150 mM NaCl, 1% Noidet P-40, and 1×protease inhibitor cocktail (P8340, manufactured by Sigma-Aldrich Corporation)) was added, and following suspension formation, a centrifugal separation treatment (20,400×g, 10 minutes, 4° C.) was used to collect the supernatant, which was then used as a disrupted cell suspension. Using Ni-NTA agarose beads (manufactured by FUJIFILM Wako Pure Chemical Corporation), the GFP-tagged protein was collected via the His tag from the disrupted cell suspension and then concentrated using spin dialysis, and a purified protein solution dissolved in a buffer containing 20 mM HEPES-KOH (pH 7.5) and 0.2% Noidet P-40 was prepared.

Prior to mixing with the GFP-tagged protein, the AF-labeled RNA was subjected to thermal denaturation by treatment at 95° C. for two minutes in the presence of either 100 mM KCl or 100 mM LiCl, and the temperature was then cooled slowly (1° C./second) until the liquid temperature reached 25° C. When RNA comprising the G4C2 sequence is cooled slowly following thermal denaturation in the presence of potassium ions, a Gq structure can be stably formed. On the other hand, in the presence of lithium ions, the Gq structure becomes unstable. RNA comprising the A4U2 sequence and RNA comprising the UG sequence do not form a Gq structure.

The disrupted cell suspension or purified protein solution described above was mixed with the AF-labeled RNA and either KCl or LiCl to prepare a reaction solution. This reaction solution was incubated for 5 minutes, and then subjected to FCCS measurement. The FCCS was conducted using a confocal microscope (LSM 510 Meta+ConfoCor3, manufactured by Carl Zeiss AG) and a water immersion objective lens (C-Apochromat 40×/1.2NA W. UV-VIS-IR, manufactured by Carl Zeiss AG). Curve fitting analysis of the acquired autocorrelation function was performed using ZEN software (from Carl Zeiss AG) using a model for two-component 3D diffusion involving a one-component exponential blinking fraction, as shown in formula (1) below.

$$[ Numerical formula 1 ]$$

$$G ( τ ) = 1 + [ 1 + \frac{T}{1 - T} exp ( - \frac{τ}{{τ}_{T}} ) ] \frac{1}{N} {\sum }_{i}^{2} {F}_{i} [ {( 1 + \frac{τ}{{τ}_{D , i}} )}^{- 1} {( 1 + \frac{τ}{{s}^{2} {τ}_{D , i}} )}^{- \frac{1}{2}} ] \qquad ( 1 )$$

In the formula, G(τ) represents an autocorrelation function or cross-correlation function of the time interval τ. Further, τ_{D,i} represents the diffusion time (i=1 or 2). Fi represents the fraction of the component (i=1 or 2). N represents the average number of particles in the confocal detection volume. T and T_{T} represent the exponential blinking fraction and the interval thereof respectively (these values are both zero for a cross-correlation function). Moreover, s represents the structural parameter determined by analyzing standard fluorescent dyes (ATTO 488 and Cy5) on the same day. The relative cross-correlation amplitude (RCA) was calculated using formula (2) shown below. In formula (2), NC and NG represent the average numbers of interacting molecules and green molecules respectively.

$$[ Numerical formula 2 ]$$

$$RCA = \frac{{N}_{C}}{{N}_{G}} \qquad ( 2 )$$

For each of TDP-43 and TDP-25, two independent tests were conducted to investigate the binding properties to RNA having the G4C2 sequence, and to RNA having the A4U2 sequence.

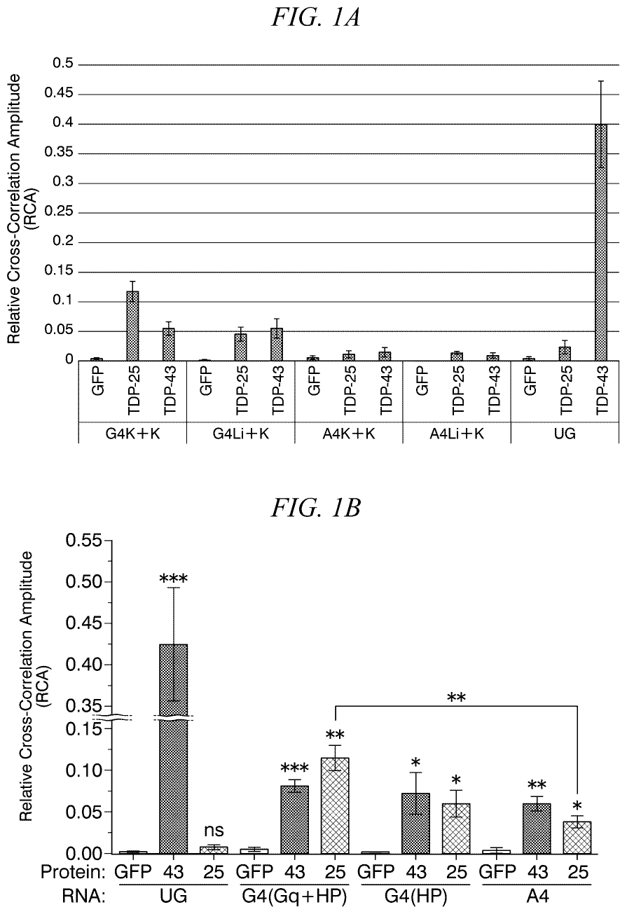

Using the disrupted cell suspensions, and AF-labeled r(G4C2)₄ that had formed a Gq structure in the presence of 100 mM KCl, AF-labeled r(G4C2)₄ prepared in the presence of 100 mM LiCl, AF-labeled r(A4U2)₄ prepared in the presence of 100 mM KCl, AF-labeled r(A4U2)₄ prepared in the presence of 100 mM LiCl, and AF-labeled r(UG)₁₂ prepared in the presence of 100 mM KCl, the first set of results for RCA (mean value±SE) measured in the presence of 100 mM KCl are shown in FIG. 1A. In the figure, the labels “GFP”, “GFP-43” and “GFP-25” indicate the results for the disrupted cell suspension of cells expressing GFP, the disrupted cell suspension of cells expressing GFP-TDP43, and the disrupted cell suspension of cells expressing GFP-TDP25 respectively. Further, in the figure, the labels “G4K+K”, “G4Li+K”, “A4K+K”, “A4Li+K” and “UG” indicate the results for AF-labeled r(G4C2)₄ that had formed a Gq structure in the presence of KCl, AF-labeled r(G4C2)₄ prepared in the presence of LiCl, AF-labeled r(A4U2)₄ prepared in the presence of KC, AF-labeled r(A4U2)₄ prepared in the presence of LiCl, and AF-labeled r(UG)₁₂ prepared in the presence of KCl respectively. These results confirmed that the AF-labeled r(UG)₁₂ exhibited a high RCA with GFP-TDP43, confirming binding of the two, but exhibited no binding to GFP-TDP25. Further, the AF-labeled r(A4U2)₄ exhibited a small RCA with both GFP-TDP43 and GFP-TDP25, regardless of the type of cation, indicating no binding could be confirmed. In contrast, in the case of the AF-labeled r(G4C2)₄, binding to both GFP-TDP43 and GFP-TDP25 was confirmed in the presence of either potassium ions or lithium ions. In particular, the binding with GFP-TDP25 for the AF-labeled r(G4C2)₄ that had formed a Gq structure was confirmed as being superior to that of the AF-labeled r(G4C2)₄ that did not have a Gq structure.

The second set of results for RCA (n=3, mean value±SE) measured using the disrupted cell suspensions, and AF-labeled r(G4C2)₄, AF-labeled r(A4U2)₄, or AF-labeled r(UG)₁₂ are shown in FIG. 1B. In the figure, the labels “GFP”, “43” and “25” indicate the results for the disrupted cell suspension of cells expressing GFP, the disrupted cell suspension of cells expressing GFP-TDP43, and the disrupted cell suspension of cells expressing GFP-TDP25 respectively. Further, in the figure, the labels “G4(Gq+HP)”, “G4(HP)”, “A4” and “UG” indicate the results when AF-labeled r(G4C2)₄ that had formed a Gq structure in the presence of KCl, AF-labeled r(G4C2)₄ prepared in the presence of LiCl, AF-labeled r(A4U2)₄ prepared in the presence of KCl, and AF-labeled r(UG)₁₂ prepared in the presence of KCl respectively were used. These results confirmed that for each of the AF-labeled r(G4C2)₄ that had formed a Gq structure, the AF-labeled r(G4C2)₄ that did not have a Gq structure, and the AF-labeled r(A4U2)₄, both binding with GFP-TDP43 and binding with GFP-TDP25 occurred. The binding with GFP-TDP43 exhibited no significant differences across the three disrupted cell suspensions, but the binding with GFP-TDP25 was significantly higher for the AF-labeled r(G4C2)₄ that had formed a Gq structure than for the AF-labeled r(A4U2)₄.

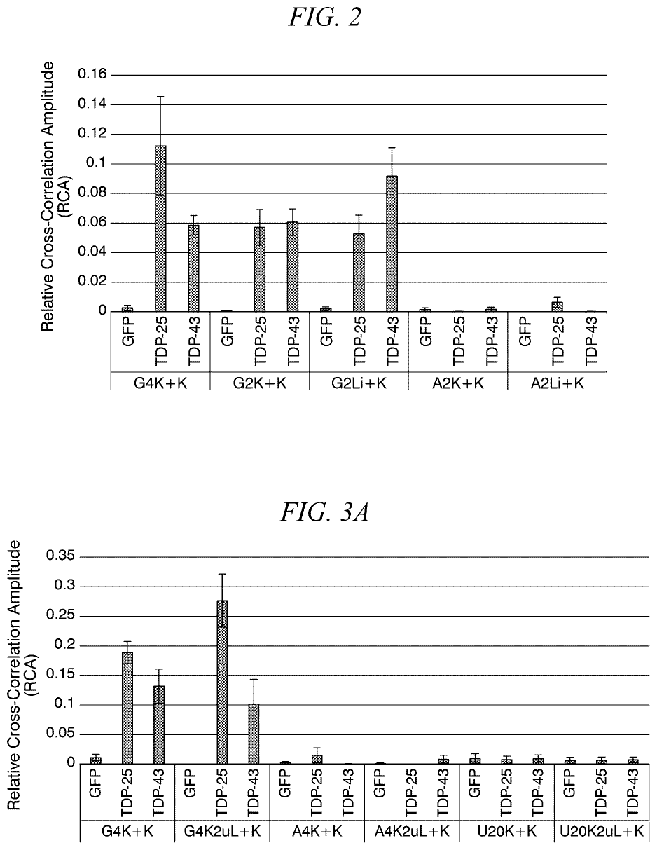

The results for RCA (mean value±SE) measured in the presence of 100 mM KCl using the disrupted cell suspensions, and AF-labeled r(G4C2)₄ that formed a Gq structure in the presence of KCl, AF-labeled r(G4C2)₂ that formed a Gq structure in the presence of KCl, AF-labeled r(G4C2)₂ prepared in the presence of LiCl, AF-labeled r(A4U2)₂ prepared in the presence of KCl, or AF-labeled r(A4U2)₂ prepared in the presence of LiCl are shown in FIG. 2. In the figure, the labels “GFP”, “TDP-43”, “TDP-25”, “G4K+K” have the same meanings as FIG. 1A. Further, the labels “G2K+K”, “G2Li+K”, “A2K+K” and “A2Li+K” indicate the results obtained using the AF-labeled r(G4C2)₂ that formed a Gq structure in the presence of KCl, the AF-labeled r(G4C2)₂ prepared in the presence of LiCl, the AF-labeled r(A4U2)₂ prepared in the presence of KCl, and the AF-labeled r(A4U2)₂ prepared in the presence of LiCl respectively. In a similar manner to the AF-labeled r(G4C2)₄, the AF-labeled r(G4C2)₂ was confirmed as exhibiting binding to both GFP-TDP43 and GFP-TDP25.

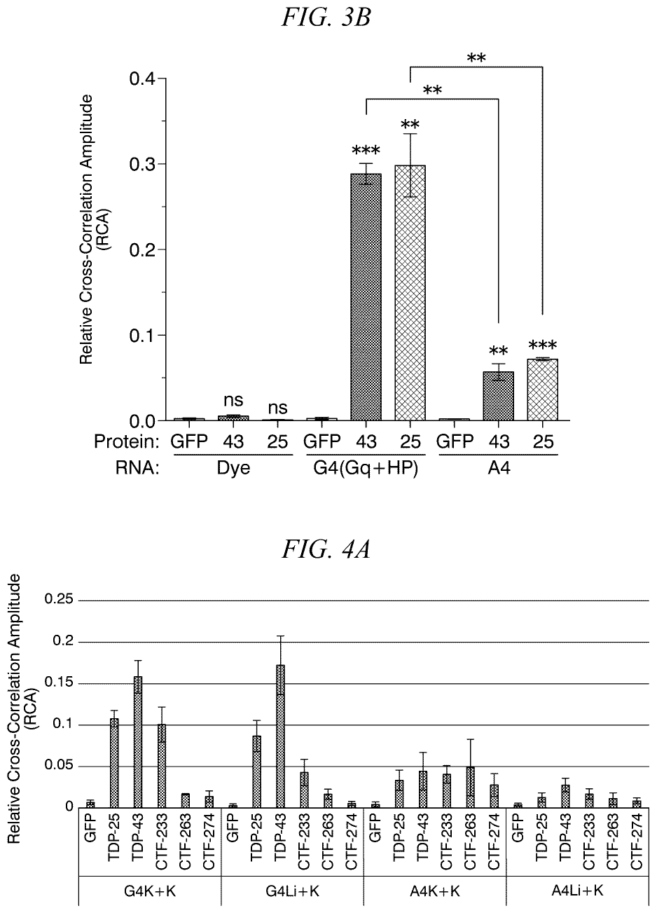

The results for RCA (mean value±SE) measured in the presence of 100 mM KCl using the purified protein solutions, and AF-labeled r(G4C2)₄ that formed a Gq structure in the presence of KCl, AF-labeled r(A4U2)₄ prepared in the presence of KCl, or AF-labeled r(U)₂₀ prepared in the presence of KCl are shown in FIG. 3A. In the figure, the labels “G4K+K”, “G4K2 uL+K”, “A4K+K”. “A4K2 uL+K”, “U20K+K” and “U20K2 uL+K” indicate the results obtained using the AF-labeled r(G4C2)₄ that formed a Gq structure in the presence of KCl, twice the amount of the AF-labeled r(G4C2)₄ compared with “G4K+K”, the AF-labeled r(A4U2)₄ prepared in the presence of KCl, twice the amount of the AF-labeled r(A4U2)₄ compared with “A4K+K”, the AF-labeled r(U)₂₀ prepared in the presence of KCl, and twice the amount of the AF-labeled r(U)₂₀ compared with “U20K+K” respectively. As shown in FIG. 3A, even when purified protein solutions were used, the AF-labeled r(G4C2)₄ was confirmed as binding to both GFP-TDP43 and GFP-TDP25, regardless of the type of cation. The purified GFP-TDP43 and the purified GFP-TDP25 were confirmed as binding directly with the AF-labeled r(G4C2)₄, without any other linking substance or the like.

The results for RCA (n=3, mean value±SE) measured in the presence of 100 mM KCl using the purified protein solutions, and AF-labeled r(G4C2)₄ that formed a Gq structure in the presence of 100 mM KCl, AF-labeled r(A4U2)₄ prepared in the presence of 100 mM KCl, or the AF dye alone are shown in FIG. 3B. In the figure, the labels “GFP”, “43”, “25”, “G4(Gq+HP)” and “A4” have the same meanings as FIG. 1B, and the label “Dye” indicates the results obtained when using only the AF dye. As shown in FIG. 3B, the AF-labeled r(G4C2)₄ and the AF-labeled r(A4U2)₄ were both confirmed as binding to both GFP-TDP43 and GFP-TDP25. The binding strength to the GFP-TDP43 and GFP-TDP25 was significantly stronger for the AF-labeled r(G4C2)₄ that formed a Gq structure than the AF-labeled r(A4U2)₄.

In order to investigate whether r(G4C2)₄ binds to any region of GFP-TDP43 and GFP-TDP25, similar RCA measurements to those described above were conducted using disrupted cell suspensions of cells expressing not only GFP-TDP43 and GFP-TDP25, but also GFP-CTF233, GFP-CTF263 and GFP-CTF274. GFP-CTF233, GFP-CTF263 and GFP-CTF274 are proteins in which a GFP tag and a His tag have been added to CTF233 (amino acid region 233 to 414 of TDP-43), CTF263 (amino acid region 263 to 414 of TDP-43), and CTF274 (amino acid region 274 to 414 of TDP-43) respectively.

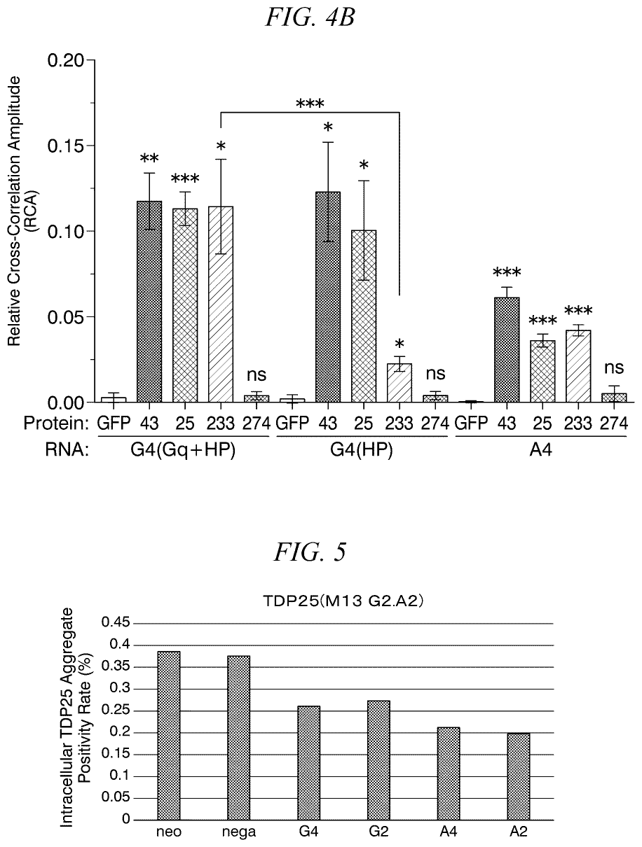

The first set of measurement results (mean value±SE) are shown in FIG. 4A. In the figure, the labels “G4K+K”, “G4Li+K”, “A4K+K” and “A4Li+K” have the same meanings as FIG. TA. The results confirmed that AF-labeled r(G4C2)₄ exhibited binding to GFP-CTF233 regardless of the type of cation, but did not bind to GFP-CTF263 or GFP-CTF274. These results suggest that in order for RNA comprising the G4C2 sequence to bind to TDP-43, the amino acid region 220 to 262 of TDP-43 is necessary.

The second set of measurement results (n=3, mean value±SE) are shown in FIG. 4B (*: P<0.05, **: P<0.01, ***: P<0.001, and ns: p>0.05). In the figure, the labels “GFP”, “43”, “25”, “G4(Gq+HP)”, “G4(HP)” and “A4” have the same meanings as FIG. 1B. Further, in the figure, the labels “233” and “274” indicate the results obtained using GFP-CTF233 and GFP-CTF274 respectively. The results confirmed that the AF-labeled r(G4C2)₄ that formed a Gq structure (labeled “G4(Gq+HP)” in the figure), the AF-labeled r(G4C2)₄ that did not form a Gq structure (labeled “G4(HP)” in the figure), and the AF-labeled r(A4U2)₄ (labeled “A4” in the figure) each exhibited binding to TDP-43 (labeled “43” in the figure), TDP-25 (labeled “25” in the figure) and GFP-CTF233 (labeled “233” in the figure), but did not bind to GFP or GFP-CTF274 (labeled “274” in the figure). The binding with GFP-CTF233 was significantly higher for the AF-labeled r(G4C2)₄ that had formed a Gq structure than for the AF-labeled r(G4C2)₄ that did not form a Gq structure.

### Example 2

RNA having a G4C2 sequence or RNA having an A4U2 sequence and GFP-TDP43 were expressed in mouse neuroblastoma Neuro2a cells, and the effect on GFP-TDP43 aggregate formation was investigated.

Specifically, first, RNA prepared by linking M13 sequences derived from a bacteriophage to the 5′-side and 3′-side of r(G4C2)₄, or more specifically, RNA (r(M13-G4)) prepared by linking GUAAAACGACGGCCAGU (SEQ ID NO. 1) to the 5′-side of r(G4C2)₄, and linking GAGCUCACCUGUGUGAAAUUGUUAUCCGCUCUU (SEQ ID NO. 2) to the 3′-side of r(G4C2)₄ was incorporated in a plasmid that transcribes such RNAs downstream from the human H1 promoter, thus preparing an expression vector for the RNA. In a similar manner, expression vectors were also prepared for RNAs (r(M13-G2), r(M13-A4) and r(M13-A2)) prepared by linking the base sequence of SEQ ID NO. 1 to the 5′-side and linking the base sequence of SEQ ID NO. 2 to the 3′-side of each of r(G4C2)₂, r(A4U2)₄ and r(A4U2)₂ respectively. Moreover, an expression plasmid for RNA comprising the M13 sequence (r(M13N)) (GUAAAACGACGGCCAGUGAGCUCACCUGUGUGAAAUUGUUAUCCGCUCUU: SEQ ID No. 3) was also prepared in a similar manner as a control. Cells with the introduced r(M13N) expression plasmid were subjected to reverse transcription PCR (RT-PCR), and the expression of r(M13N) was confirmed.

The pHS-GFP-TDP25 used in Example 1, and the r(M13-G4) expression plasmid, the r(M13-G2) expression plasmid, the r(M13-A4) expression plasmid, the r(M13-A2) expression plasmid, the r(M13N) expression plasmid, or an empty vector (a plasmid not incorporating r(M13N)) were each transfected into Neuro2a cells using Lipofectamine 2000 (manufactured by Thermo Fisher Scientific Inc.), and following culturing for 24 hours, a confocal microscope was used to measure the intracellular aggregate positivity rate (%) (the proportion of cells in which GFP-TDP25 aggregates have formed inside the cells relative to the total number of cells).

The measurement results for the intracellular aggregate positivity rate (%) are shown in FIG. 5. In the figure, the labels “neo”, “nega”, “G4”, “G2”, “A4” and “A2” indicate cells transfected with the empty vector, the r(M13N) expression plasmid, the r(M13-G4) expression plasmid, the r(M13-G2) expression plasmid, the r(M13-A4) expression plasmid, and the r(M13-A2) expression plasmid respectively. As illustrated in FIG. 5, the intracellular aggregate positivity rate (%) for the cells transfected with the r(M13N) expression plasmid exhibited no difference from that of the cells transfected with the empty vector. In contrast, in the cells transfected with r(M13-G4), r(M13-G2), r(M13-A4) and r(M13-A2), the intracellular aggregate positivity rate was clearly reduced compared with that of the cells transfected with the empty vector, confirming that these RNAs have the effect of inhibiting TDP-25 aggregate formation.

RNAs prepared by adding the mKeima sequence instead of the M13 sequence (for example, r(mKeima-G4) and the like) were also investigated, by creating expression plasmids in the same manner as that described above, performing transfection into Neuro2a cells together with pHS-GFP-TDP25, and then investigating the intracellular aggregate positivity rate (%). The RNAs with an added mKeima sequence were prepared by linking UCUUUGCACGAUGGAAUG (SEQ ID NO. 4) to the 5′-side of each of r(G4C2)₄, r(G4C2)₂, r(A4U2)₄, and r(A4U2)₂, and linking GAGCUCAAGCAUCCAAGGCAACUGUUUUUUUA (SEQ ID NO. 5) to the 3-side of r(G4C2)₄.

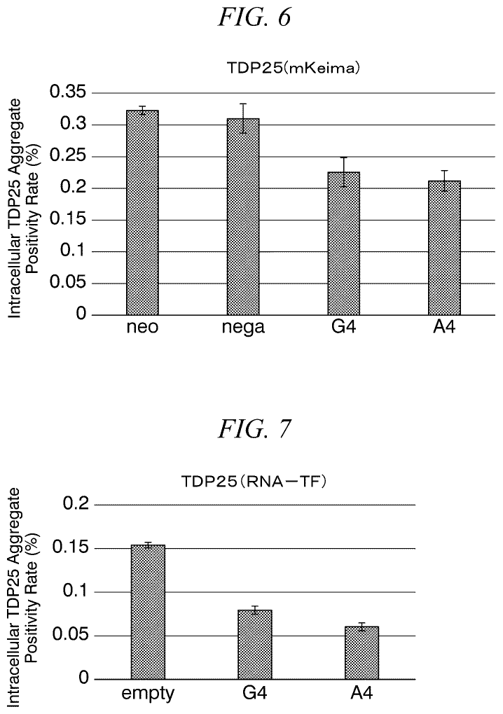

The measurement results for the intracellular aggregate positivity rate (%) are shown in FIG. 6. In the figure, the labels “neo”, “G4” and “A4” have the same meanings as FIG. 5, and “nega” indicates cells transfected with an r(mKeima) expression plasmid. Even when the tag sequence is the mKeima sequence, in a similar manner to that observed for the M13 sequence, the cells transfected with r(mKeima-G4) and r(mKeima-A4) exhibited a significantly reduced intracellular aggregate positivity rate compared with the cells transfected with the empty vector.

Using Lipofectamine 2000, r(G4C2)₄ and r(A4U2)₄ were transfected into Neuro2a cells together with pHS-GFP-TDP25, and the intracellular aggregate positivity rate (%) was investigated in the same manner as described above. The measurement results for the intracellular aggregate positivity rate (%) are shown in FIG. 7. In the figure, the labels “G4” and “A4” have the same meanings as FIG. 5, and “empty” indicates the results for cells into which no RNA was transfected. It was confirmed that even in those cases where RNA having the G4C2 sequence or RNA having the A4U2 sequence was transfected directly into the cells, aggregation of TDP-25 was able to be inhibited.

Further, an investigation was conducted as to whether the aggregation inhibitory effect of r(G4C2)₄ or the like manifested in the case of the TDP43CS variant (a C173S/C175S amino acid substituted variant) (Non-Patent Document 7), which among the various aggregating variants of TDP-43, is a variant that lacks the capabilities of nuclear localization and signal sequence export. Specifically, pHS-GFP-TDP43CS was first produced in a similar manner to that described for pHS-GFP-TDP25. Subsequently, the pHS-GFP-TDP43CS, and the r(M13-G4) expression plasmid, the r(M13-A4) expression plasmid or the r(M13N) expression plasmid were transfected into 293 cells using Lipofectamine 2000, and after culturing for 24 hours, a confocal microscope was used to measure the intracellular aggregate positivity rate (%). In a similar manner, 293 cells were transfected with only pHS-GFP-TDP43CS, and following culturing, the intracellular aggregate positivity rate (%) was measured. Culturing of the 293 cells was conducted in the same manner as the culturing of the Neuro2a cells.

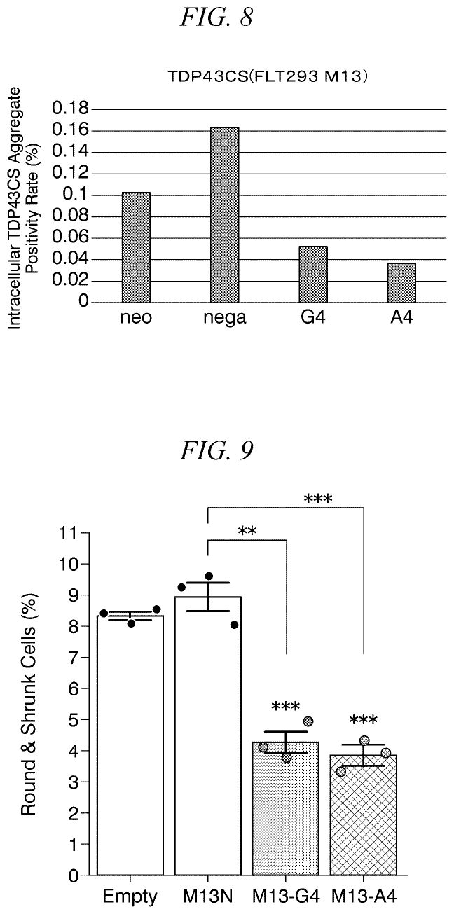

The measurement results for the intracellular aggregate positivity rate (%) are shown in FIG. 8. The cells transfected with r(M13-G4) or r(M13-A4) exhibited a significantly reduced intracellular aggregate positivity rate compared with the cells transfected with the empty vector, confirming that these RNAs were also able to inhibit the formation of aggregates of TDP43CS.

Moreover, the total RNA was recovered from Neuro2a cells expressing r(G4C2)₄ or r(A4U2)₄ from downstream of the H1 promoter, and when the mRNA and ncRNA expression were investigated by RNA-seq, no significant change in the transcriptome was observed.

Cytoplasmic mislocalization of TDP-43 is cytotoxic. Accordingly, an investigation was conducted as to whether cytotoxicity caused by the mislocalization of TDP-43 could be ameliorated by expressing r(M13-G4) or r(M13-A4). Specifically, the proportion (%) of rounded and shrunk cells among the total cell count expressing TDP-43 was measured. First, 293 cells were transfected with pHS-GFP-TDP43CS, and the r(M13-G4) expression plasmid, the r(M13-A4) expression plasmid or the r(M13N) expression plasmid using Lipofectamine 2000, and after culturing for 24 hours, the cell morphology was inspected using a confocal microscope. Further, 293 cells were also transfected with only the pHS-GFP-TDP43CS using Lipofectamine 2000, and the cell morphology was inspected in a similar manner. For each cell group, the results of measuring the proportion (%) of cells having a rounded shape or shrunk shape relative to the total cell count (n=3, mean value±SE) are shown in FIG. 9 (**: p<0.01, ***: p<0.001). The results revealed that in the cells transfected with r(M13-G4) or r(M13-A4), the number of rounded and shrunk cells decreased significantly compared with the cells transfected with only the pHS-GFP-TDP43CS (labeled “Empty” in the figure). Based on these results, it was clear that r(G4C2)₄ and r(A4U2)₄ were capable of reducing the cytotoxicity caused by cytoplasmic mislocalization of TDP-43.

1. An aggregation inhibitor that inhibits aggregation of TDP-43 or a partial protein thereof, the aggregation inhibitor comprising, as an active ingredient, an RNA molecule having either a G4C2 sequence, which is a base sequence represented by GGGGCC, or an A4U2 sequence, which is a base sequence represented by AAAAUU.

2. The aggregation inhibitor according to claim 1, wherein the RNA molecule comprises RNA in which from one to eight of the G4C2 sequences are linked.

3. The aggregation inhibitor according to claim 1, wherein the RNA molecule comprises RNA in which from one to eight of the A4U2 sequences are linked.

4. The aggregation inhibitor according to claim 1, wherein the RNA molecule includes both the G4C2 sequence and the A4U2 sequence.

5. (canceled)

6. (canceled)

7. (canceled)

8. A method for inhibiting aggregation of TDP-43 or a partial protein thereof, comprising administering the aggregation inhibitor according to claim 1 to a subject.

9. A method for prevention or treatment of a neurodegenerative disease, comprising administering the aggregation inhibitor according to claim 1 to a subject.

10. The method according to claim 9, wherein the neurodegenerative disease is amyotrophic lateral sclerosis or frontotemporal lobar degeneration.
